(19) United States
(12) Patent Application Publication
(10) Pub. No.: US 2019/0358377 A1
(43) Pub. Date: Nov. 28, 2019

(54) CIRCULATORY ASSISTANCE SYSTEM

(71) Applicant: Association Marie Lannelongue, Le Plessis Robinson (FR)

(72) Inventor: Marc-Antoine Isorni, Versailles (FR)

(73) Assignee: Association Marie Lannelongue, Le Plessis Robinson (FR)

(21) Appl. No.: 16/345,355

(22) PCT Filed: Oct. 27, 2017
(86) PCT No.: PCT/EP2017/077681

§ 371 Date: May 24, 2019

(30) Foreign Application Priority Data
Oct. 28, 2016 (FR) 1660511

(51) Int. Cl. A61M 1/10; A61M 1/12; F01C 13/04; F04C 18/22

(52) U.S. Cl. CPC A61M 1/1012; F04C 18/22; F01C 13/04; A61M 1/122

(57) ABSTRACT

The invention relates to a circulatory assistance system comprising: a rotor in the shape of a Reuleaux triangle; and a camshaft received in a housing of the rotor and designed to rotate the rotor about an axis of rotation. The inner walls of the housing of the rotor are in contact at all points with the outer surface of the cam such that the assembly formed by the cam and the rotor does not have a clearance volume.

### FIELD OF THE INVENTION

The invention relates to the field of implantable prostheses, and more particularly of circulatory assistance systems such as heart pumps.

### TECHNOLOGICAL BACKGROUND

Heart failure is a major public health problem in developed countries to the point of affecting approximately 5.7 million Americans with an incidence of 670,000 patients a year. Its impact in terms of morbidity and mortality is estimated at 300,000 deaths and 2.4-3.5 million hospitalizations per year.

1 to 10% of these patients suffer from an advanced form called “terminal” form where the heart is no longer sufficient to ensure the circulatory function of the body and leads to a multiple organ dysfunction. Medical treatments represent then only a palliative therapeutic option while curative treatments are limited.

Nowadays, the reference treatment is the heart transplantation. However, it cannot meet the needs of this population due to lack of a sufficient number of organ donors. Circulatory assistance systems have therefore found their place by offering a temporary or permanent alternative to the graft.

Artificial hearts (for example comprising a centrifugal pump, heterotopic pneumatic ventricles, electromechanical implantable ventricles, continuous-flow pumps or total artificial hearts) implantable into a human to assist or fully replace a failing heart, have already been proposed. An artificial heart is a prosthesis made from synthetic and/or biological materials, used in the treatment of people with irreversible heart failure.

However, to date, none of the proposed artificial hearts has been able to give complete satisfaction because none meets the very severe conditions which must be absolutely fulfilled by an artificial heart at the risk of failure.

Among these conditions, it is first necessary that the space requirement of the artificial heart is sufficiently reduced so that the latter can be implanted and carried by the patient without leading to rejection phenomena and without constituting a very restrictive discomfort.

Another condition is that the circulatory assistance system comprises a portion ensuring systemic circulation and a portion ensuring pulmonary circulation each having a pulsatile suctioned flow and a pulsatile discharged flow, i.e. the flows must both go through a maximum and then cancel each other.

In addition, the suction loss must be low in order to prevent hemolysis of the blood when the latter is depressed, even very little. For the same purpose, it is necessary to avoid the turbulences that are likely to produce pressure changes, in particular depressions. The respiratory assistance system must therefore preserve the laminar streams and avoid iso-volumic barometric stresses.

It is also necessary to avoid the risks of thrombosis (clot formation) by limiting the areas of platelet accumulation and the number of parts in contact with blood as well as the friction areas likely to damage the red blood cells and therefore to induce a hemolysis.

Finally, the respiratory assistance system must be robust.

It has therefore been proposed in document FR 2 389 382 a heart pump comprising a rotary piston, a straight section of which is a trochoid, which is rotatably movable in a body. This particular shape of the rotor indeed makes it possible to obtain a pump having a uniform rotational speed, whose suctioned and discharged flows have a pulsating regime and whose space requirement allows its implantation in the thoracic cavity of a patient. However, this device has dead spaces in which blood is likely to stagnate and form thrombi as well as friction areas conducive to the hemolysis of red blood cells.

### SUMMARY OF THE INVENTION

An object of the invention is therefore to propose a circulatory assistance system implantable into a living being limiting the risks of hemolysis of blood and thrombosis, which is also pulsatile, simple to produce and easy to adapt in order to be possible to adjust it in the body of a patient regardless of his morphology or age.

For this, the invention proposes a circulatory assistance system comprising:
a rotor comprising an outer surface defining a curve having the shape of a Reuleaux triangle and a housing delimited by inner walls, and
a camshaft comprising a central shaft on which a cam is securely fixed, the camshaft being housed in the housing of the rotor and configured to rotatably drive the rotor about an axis of rotation, the axis of rotation of the camshaft being offset from a center of symmetry of the rotor.

An outer surface of the cam defines a shell substantially complementary to the inner walls of the housing so that said inner walls are in contact at all points with the outer surface of the cam and that the assembly formed by the cam and the rotor is devoid of dead space.

Such a system is advantageously completed by the different following characteristics taken alone or in all their technically possible combinations:
the outer surface of the cam is smooth;
the outer surface of the cam has a circular shape;
the system further comprises:
   a stator in which is formed a cavity housing the rotor, a side wall of said cavity having a trochoidal shape defined by the curve traversed by apexes of the rotor corresponding to the vertices of the Reuleaux triangle, the cavity being divided into a first chamber and a second chamber symmetrical with respect to a plane passing through the axis of rotation of the camshaft,
   a first intake port and a first discharge port each opening into the first chamber, and
   a second intake port and a second discharge port each opening into the second chamber,
   the first intake port and the second intake port on the one hand, and the first discharge port and the second intake port on the other hand, being symmetrical with respect to the plane;
in the proposed system,
   each apex of the rotor has a predefined height along a direction parallel to the axis of rotation,
   the first and second intake ports and the first and second discharge ports each have a height along the direction parallel to the axis of rotation,
   the height of each port being smaller than the predefined height of each apex of the rotor;
the stator further has a lower wall and an upper wall substantially perpendicular to the axis of rotation and each port extends at a distance from the lower wall and/or from the upper wall of the stator so that the side wall of the cavity is substantially continuous at the lower wall and/or at the upper wall of the stator;
each port has a substantially rectangular section;
for one of the chambers, the distance between an area of the side wall immediately downstream of the intake port and an area of said side wall immediately upstream of the discharge port, is greater than the sum of a distance between two apexes of the rotor and of a maximum dimension of an activated platelet, so as to form the clearance between the rotor and the stator;
the stator further has an upper wall and a lower wall connecting the side wall of the cavity, a minimum distance between the upper wall and the rotor and between the lower wall and the rotor being greater than a maximum height of the rotor;
the lower wall and the upper wall have surface irregularities configured to locally create a turbulent stream in the system;
the rotor extends at a distance of at least 7 micrometers from the stator walls.

### SHORT DESCRIPTION OF THE DRAWINGS

Other characteristics, objects and advantages of the present invention will become more apparent upon reading the following detailed description, and with reference to the appended drawings given by way of non-limiting examples and in which:

FIG. 1 is a top view of an exemplary embodiment of a circulatory assistance system according to the invention which illustrates a phase of intake in a first ventricular chamber and a phase of draining from a second ventricular chamber,

FIG. 2 is a top view of the circulatory assistance system of FIG. 1 which illustrates a neutral time of the first ventricular chamber,

FIG. 3 is a top view of the circulatory assistance system of FIG. 1 which illustrates a phase of draining from the first ventricular chamber and a phase of intake in the second ventricular chamber,

FIG. 4 is a top view of the circulatory assistance system of FIG. 1 which illustrates a neutral time of the second ventricular chamber,

FIG. 5 is a partial perspective view of an exemplary embodiment of a circulatory assistance system according to the invention, on which an intake port and a discharge port are visible in transparency,

FIG. 6 is a partial top view of an exemplary embodiment of a circulatory assistance system according to the invention, on which is illustrated a shunt area at an intake port and a discharge port of the system,

FIG. 7 is a top view of an exemplary embodiment of a front face of a circulatory assistance system according to the invention,

FIG. 8 is a sectional view of the front face of FIG. 7, and

FIG. 9 is a partial sectional view according to the height of the rotor of an exemplary embodiment of a circulatory assistance system according to the invention.

### DETAILED DESCRIPTION OF AN EMBODIMENT

A circulatory assistance system 1 according to the invention comprises a rotor 10, a camshaft 20 and a stator 30.

The rotor 10 comprises an outer surface defining a curve having the shape of a Reuleaux triangle, and a housing delimited by inner walls.

The Reuleaux triangle is a curve of constant width, i.e. a curve all of whose diameters have the same length. More precisely, the Reuleaux triangle is a closed plane curve whose width, measured by the distance between two opposite parallel straight lines tangent thereto, is the same whatever the orientation of these straight lines. The rotor 10 thus has three apexes 12 corresponding to the vertices of the Reuleaux triangle separated by three curved surfaces. There is also meant by radius R of the rotor 10, the distance between the center of symmetry of the rotor 10 and an apex 12 of the rotor 10.

The camshaft 20 comprises a central shaft 22 on which a cam 24 is securely fixed. The camshaft 20 is housed in the housing of the rotor 10 and is configured to rotatably drive the rotor 10 about an axis of rotation X corresponding to the axis of extension of the central shaft 22. The rotor 10 is fixed on the cam 24 and the cam 24 is configured so that the center of symmetry C of the rotor 10 is offset from the axis of rotation X of the central shaft 22 by a distance e corresponding to the eccentricity of the cam 24.

The central shaft 22 of the camshaft 20 is configured to be connected to a motor capable of rotatably driving the central shaft 22 at a determined speed. The rotational speed of the central shaft 22 can in particular be adjusted according to the desired flow in the circulatory assistance system 1, which depends inter alia on the body surface of the patient.

An outer surface 25 of the cam 24 defines a shell substantially complementary to the inner walls of the housing so that the inner walls 15 of the rotor 10 are in contact at all points with the outer surface 25 of the cam 24 and that the assembly formed by the cam 24 and the rotor 10 is devoid of dead space. In other words, the shape of the outer surface 25 of the cam 24 is complementary to the shape of the inner walls of the housing of the rotor 10 and the height of the cam 24 is substantially equal to the height of the housing.

Here, it is meant by height, a dimension (in particular of the camshaft 20, of the rotor 10 or of the stator 30) along a direction substantially parallel to the axis of rotation X.

The surface continuity provides a real benefit insofar as it significantly reduces the risks of thrombosis and hemolysis in the system 1. Indeed, the blood is unlikely to stagnate between the inner walls 15 of the rotor 10 and the wall 25 of the cam 24, or to be ground between these parts during the movement of the rotor 10.

The outer surface 25 of the cam 24 and the inner walls 15 of the housing may for example be smooth, i.e. devoid of protrusion or meshing means. The Applicant has indeed noticed that the transmission of forces to the rotor 10 by simple friction between the outer surface 25 of the cam 24 and the inner walls 15 of the housing was sufficient in the case of a circulatory assistance system 1 of the heart pump type.

For example, the outer surface 25 of the cam 24 may have a circular section.

Alternatively, the cam 24 of the camshaft 20 and the rotor 10 (and possibly its central shaft 22) may be unitary, that is to say formed integrally and in one piece or fixed together by bonding.

The rotor 10 is housed in a cavity 31 formed in the stator 30.

The side wall 32 of the cavity 31 has a trochoidal shape defined by the curve traversed by the apexes 12 of the rotor 10. In other words, the apexes 12 of the rotor 10 substantially continuously sweep the side wall 32 of the cavity 31 during the rotation of the rotor 10 in the stator 30. The ratio between the radius R of the rotor 10 and the eccentricity e therefore defines the shape of the side wall 32 of the cavity 31.

The stator 30 further has an upper wall 33 and a lower wall 34 substantially parallel to each other and normal to the axis of rotation X of the camshaft 20, which extend facing an upper face 14 and a lower face 16 of the rotor 10, respectively.

The cavity 31 of the stator 30 is divided into a first ventricular chamber 35 and a second ventricular chamber 38 which extend symmetrically with respect to a first plane P1 passing through the axis of rotation X of the central shaft 22 of the camshaft 20.

In order to allow the systemic circulation and the pulmonary circulation in the circulatory assistance system 1, intake 36, 39 and discharge 37, 40 ports opening into the first chamber 35 and into the second chamber 38 are formed in the side wall 32 of the stator 30. More specifically, the first chamber 35 comprises a first intake 36 and a first discharge 37 port while the second chamber 38 comprises a second intake 39 and a second discharge 40 port. Note that the first intake port 36 and the second discharge port 40 on the one hand, and the first discharge port 37 and the second intake port 39 on the other hand, are symmetrical with respect to the first plane P1 in order to make the suctioned and discharged flows by the two pulsatile chambers. They are also symmetrical with respect to a second plane P2 which extends perpendicularly to the first plane P1 and passes through the center of rotation of the shaft.

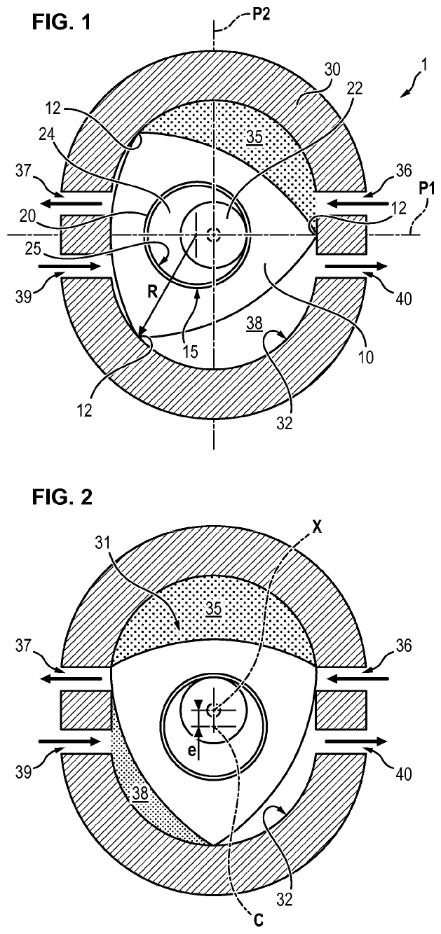

The four phases of an operating cycle of the circulatory assistance system 1 are thus illustrated in FIGS. 1 to 4. In the figures, the rotor 10 rotates in the counterclockwise direction. This is however not limiting, the rotor 10 being rotatable in the clockwise direction.

During a first phase (FIG. 1), the rotor 10 is positioned so as to release the first intake port 36 and close the first discharge port 37 of the first chamber 35 and to close the second intake port 39 and release the second discharge port 40 of the second chamber 38. The rotational movement of the rotor 10 in the counterclockwise direction therefore has the effect of suctioning a fluid through the first intake port 36 and draining a fluid by the second discharge port 40 by closing the discharge path of the first chamber 35 and the intake path of the second chamber 38.

During a second phase (FIG. 2), the rotor 10 is positioned so as to block the first intake port 36 and the first discharge port 37 and release the second intake port 39 and the second discharge port 40 of the second chamber 38. This second phase therefore corresponds to a neutral time for the first chamber 35 (zero flow). Moreover, the rotational movement of the rotor 10 in the counterclockwise direction has the effect of suctioning a fluid through the second intake port 39 and completing the drainage of the fluid through the second discharge port 40.

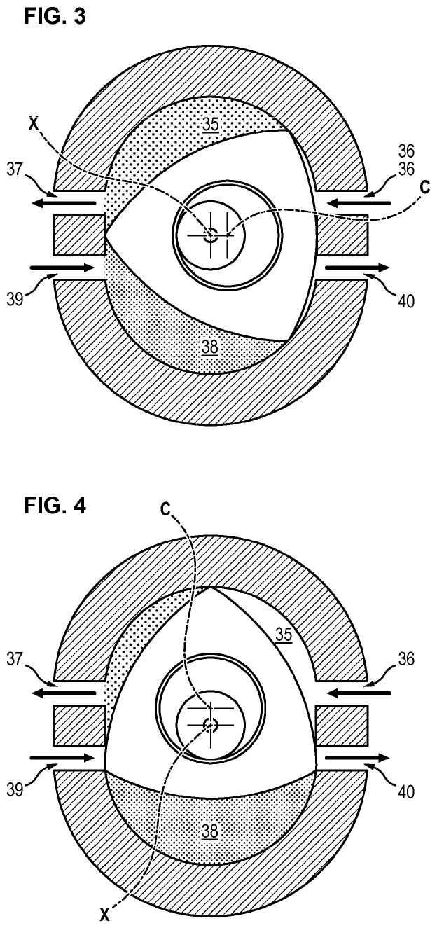

During a third phase (FIG. 3), the rotor 10 is positioned so as to keep open the second intake port 39 and close the second discharge port 40 of the second chamber 38 and to release the first discharge port 37 and close the first intake port 36 of the first chamber 35. The rotational movement of the rotor 10 in the counterclockwise direction therefore has the effect of continuing the suction of the fluid through the second intake port 39 and starting to drain the fluid suctioned during the first phase through the first discharge port 37 by closing the discharge path of the second chamber 38 and the intake path of the first chamber 35.

During a fourth phase (FIG. 4), the rotor 10 is positioned so as to block the second intake port 39 and the second discharge port 40 of the second chamber 38 and to release the first intake port 36 and keep open the first discharge port 37 of the first chamber 35. This fourth phase therefore corresponds to a neutral time for the second chamber 38 (zero flow). Moreover, the rotational movement of the rotor in the counterclockwise direction has the effect of suctioning a fluid through the first intake port 36 and completing the drainage of the fluid through the first discharge port 37.

The first, second, third and fourth phases of the cycle take place continuously and are consecutive. At the end of the fourth phase of a given cycle, a new cycle then begins by repeating the phases from the first to the fourth one.

A pulsatile circulatory assistance system 1 is therefore well obtained, in which the flow of each chamber 35, 38 passes through a maximum and is then alternately cancelled.

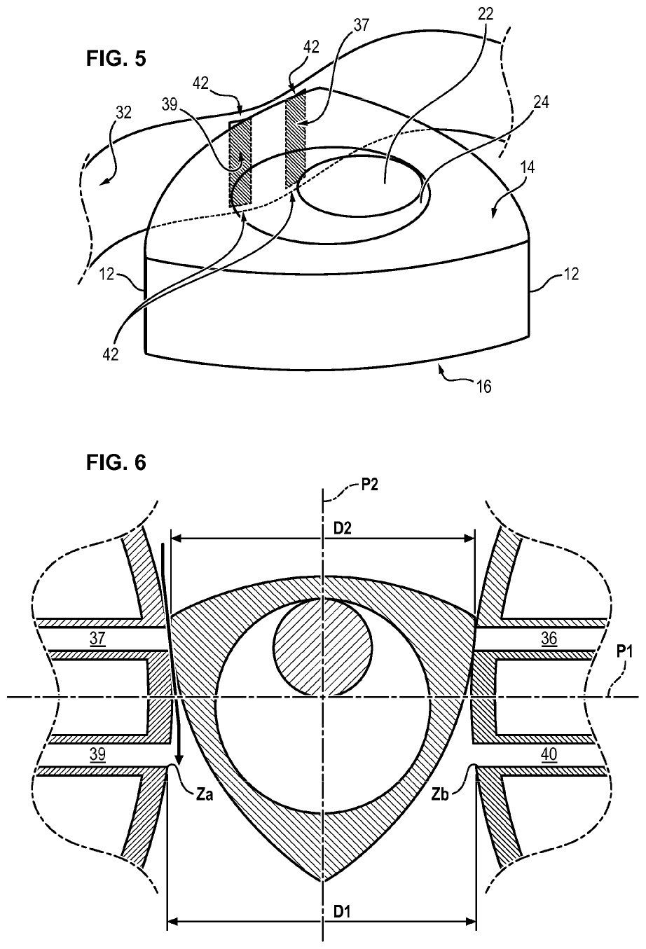

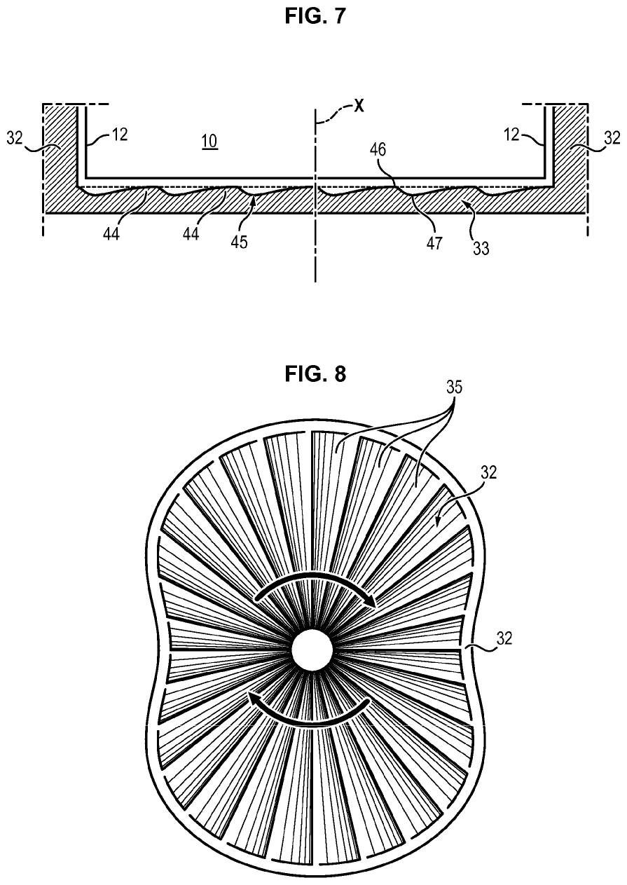

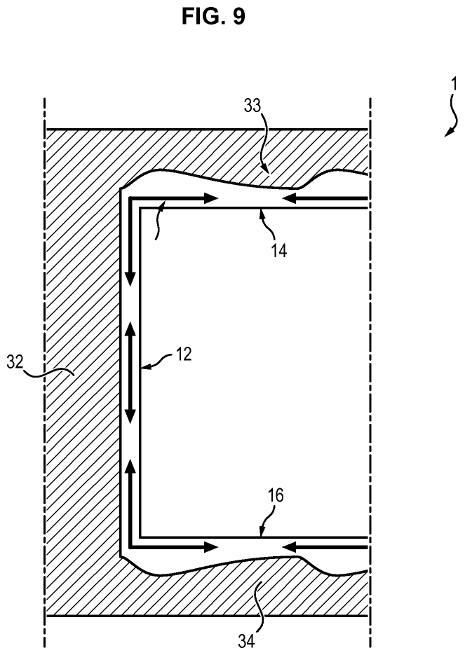

In one embodiment, each port 36, 37, 39, 40 has a rectangular section, the length of the rectangle extending along the height of the stator 30 (see FIG. 5). Such a section makes it possible to preserve a laminar flow in the system 1 even in the vicinity of the ports and thus to limit the friction forces likely to be applied to blood by maintaining constant shear stresses, thus reducing the risks of hemolysis. The dimension of the section (and more particularly its width) is moreover determined by the flow that the system 1 must provide.

In order to reduce the risks of blocking the rotor 10 in the ports 36, 37, 39, 40, the height of the ports 36, 37, 39, (corresponding to the length of their rectangular section) is smaller than the height of the apexes 12 the rotor 10 and therefore smaller than the height of the side wall 32 of the stator 30. Thus, the side wall 32 forms a continuous shell on the periphery of the cavity 31, even at the ports 36, 37, 39, 40, thus forming areas above and/or below the ports 36, 37, 39, 40 forming slides 42 for the apexes 12 of the rotor 10 preventing them from entering the ports 36, 37, 39, 40 and rotatably blocking the rotor 10.

In the exemplary embodiment illustrated in the figures, the ports 36, 37, 39, 40 extend at a distance from the upper wall 33 and from the lower wall 34 of the stator 30 so that slides 42 are formed on either side of the ports 36, 37, 39, 40.

The height of each slide 42 may be in the order of a few millimeters.

In order to limit the areas of platelet aggregation and therefore the formation of thrombi (or clots), the side wall 32 of the cavity 31 is dimensioned so as to maintain a minimum clearance (preferably over the entire height of the side wall 32) between the side wall 32 and the apexes 12 of the rotor 10 regardless of the operating phase of the system 1 (FIG. 6). Note however that, during the revolution of the rotor 10, said rotor 10 remains in contact at any time with the side wall 32 at at least one point in order to force its tilting.

This minimum clearance thus generates a wash stream making it possible to continuously stir the first chamber 35 and the second chamber 38 during rotation of the rotor 10 in the stator 30, even during the neutral times, thus greatly reducing the risks of thrombus formation.

It will be noted that the permanent passage of a stream between the first chamber 35 and the second chamber 38 during neutral times is not uncomfortable for the patient insofar as such shunt areas also exist in the human heart.

In one embodiment, the minimum clearance is greater than the dimension of an activated platelet (namely about seven micrometers) in order to make sure that the platelets are not crushed or at least stressed between the rotor 10 and the side wall 32 of the cavity 31 of the stator 30. For example, the clearance between the side wall 32 and the apexes 12 of the rotor 10 may be in the order of a few millimeters. The areas of the system 1 in which the apexes 12 of the rotor 10 are at a short distance from the side wall 32 of the cavity 31 are then less likely to undergo stresses, which significantly reduces the risk of hemolysis of the blood.

For this, the dimensions and the shape of the side wall 32 of the cavity 31 of the stator 30 are chosen so that the distance D1 between the area Za of the side wall 32 immediately downstream of the intake port 36 (respectively 39) and the area Zb immediately upstream of the discharge port 37 (respectively 40) (the upstream and the downstream being defined relative to the direction of rotation of the rotor 10 in the stator 30) is greater than the sum of the distance D2 between two apexes 12 of the rotor 10 and the dimension of an activated platelet (in the order of seven micrometers) so as to obtain the desired minimum clearance. Preferably, a rotor 10 will be chosen such that the distance D1 remains smaller than the sum of the distance D2 and a few millimeters.

It will be noted that the distance D2 is generally equal to 2.R.cos(30), where R corresponds to the radius R of the rotor 10.

Thus, FIG. 6 for example illustrates the case where the shunt area is formed at the discharge port 37 and the intake port 38, this being non-limiting, the shunt area may alternatively be made at the discharge port 40 and the intake port 39.

There is also a risk of formation of thrombus and hemolysis between the upper 33 and lower 34 walls of the stator 30 and the faces 14, 16 opposite the rotor 10 resulting from the accumulation of blood between these portions of the parts and/or stresses applied by these portions to blood. A minimum clearance can therefore also be formed between the upper 33 and lower 34 walls of the stator 30 and the faces 14, 16 opposite the rotor 10 (FIG. 9).

In one embodiment, the minimum clearance between the upper 33 and lower 34 walls of the stator 30 and the faces 14, opposite the rotor 10 is greater than the dimension of a platelet (about seven micrometers) to limit the risks of hemolysis of blood, for example in the order of one millimeter (within two micrometers).

The surface of the upper 33 and lower 34 walls of the stator 30 facing the rotor 10 may further have surface irregularities 44 configured to locally create a turbulent stream in the system 1 and thus force the evacuation of the red blood cells inevitably accumulated against said walls 33, 34. To that end, the surface irregularities 44 are chosen to transform the laminar flow of blood when approaching the lower face 16 and the upper face 14 into a turbulent stream, thereby limiting the risks of thrombus formation.

The surface irregularities 44 may in particular have the form of fins (or helixes) extending radially flaring from the axis of rotation X towards the side wall 32. The fins 44 therefore have a substantially triangular section. In one embodiment, each fin 44 has a variable height from upstream to downstream (relative to the direction of rotation of the rotor 10 in the stator 30) to promote the generation of a turbulent stream. The profile may thus have an upstream section whose thickness is increasing from the upstream edge 45 of the fin 44 up to an intermediate apex 46 and then a downstream section whose thickness decreases from the apex 12 up to the downstream edge 47 of the fin 44. The slope of the upstream section is smoother than the slope of the downstream section.

For example, the angle between the upstream edge 45 and the apex 46 of a given fin 44 is at least twice as large as the angle between this apex 12 and the downstream edge 47 of said fin 44.

The fins 44 are preferably adjacent to each other so as to form a symmetrical relief about the axis of rotation X. The upstream edge 45 of a given fin 44 therefore corresponds to the downstream edge 47 of the fin 44 extending immediately upstream.

In this way, the rotation of the rotor 10 relative to the stator 30 causes the generation of a clean stream at each fin discharging the red blood cells and thus avoiding their stagnation.

In one embodiment, the height of the surface irregularities 35 is smaller than the size of an activated platelet, namely smaller than seven micrometers, in order to prevent stagnation of said platelets between the irregularities. Thus, in the case of surface irregularities 35 comprising fins 44, the height between the apex 46 of a given fin 44 and the downstream edge 47 of this fin 44 is preferably less than seven micrometers.

The dimension of the circulatory assistance system 1 depends on the anatomy of the patient intended to receive the system 1, and in particular on his cardiac output.

The cardiac output of a patient can be determined for example according to his body surface area. Such a determination being known to those skilled in the art, it will not be detailed here.

The knowledge of the cardiac output thus makes it possible to determine, on the one hand, the dimensions of the cavity 31 and of the rotor 10, and on the other hand the rotational speed of the camshaft 20.

The dimensions of the cavity 31 are determined in practice from the eccentricity e of the cam 24, from the radius R of the rotor 10 and from the height of the stator 30. Particularly, the R/e ratio makes it possible to define the trochoidal curve of the cavity 31 and thus the shape of each chamber 35, 38 of the stator 30 while the height of the stator 30 defines the volume of the chambers. The ratio R/e may in particular be comprised between 5.0 and 6.5.

Indeed, the geometry of the trochoidal shape of the cavity 31 is defined by the following trigonometric formulae, which depend on the eccentricity e of the cam 24 and on the radius R of the rotor 10:

x=e×cos(3α)+R×cos(α)

y=e×sin(3α)+R×sin(α)

For example, the ratio between the radius R of the rotor 10 and the eccentricity e may be equal to 5.678.

The system 1 is therefore simple and comprises few movable parts (a rotor 10 and a camshaft 20), which makes it reliable and allows greatly reducing the frictions and therefore the energy requirements and the risks of thrombosis and hemolysis.

1. A circulatory assistance system comprising:
a rotor comprising an outer surface defining a curve having the shape of a Reuleaux triangle and a housing delimited by inner walls,
a camshaft comprising a central shaft to which a cam is securely fixed, the camshaft being housed in the housing of the rotor and configured to rotatably drive the rotor about an axis of rotation, the axis of rotation of the camshaft being offset from a center of symmetry of the rotor, and
the circulatory assistance system wherein an outer surface of the cam defines a shell substantially complementary to the inner walls of the housing so that said inner walls are in contact at all points with the outer surface of the cam and that the assembly formed by the cam and the rotor is devoid of dead space.

2. The circulatory assistance system according to claim 1, wherein the outer surface of the cam is smooth.

3. The circulatory assistance system according to claim 1, wherein the outer surface of the cam has a circular shape.

4. The circulatory assistance system according to claim 1, further comprising:
a stator in which is formed a cavity housing the rotor, a side wall of said cavity having a trochoidal shape defined by the curve traversed by apexes of the rotor corresponding to the vertices of the Reuleaux triangle, the cavity being divided into a first chamber and a second chamber symmetrical with respect to a plane passing through the axis of rotation of the camshaft,
a first intake port and a first discharge port each opening into the first chamber, and
a second intake port and a second discharge port each opening into the second chamber,
the first intake port and the second intake port on the one hand, and the first discharge port and the second intake port on the other hand, being symmetrical with respect to on the plane.

5. The circulatory assistance system according to claim 4, wherein
each apex of the rotor has a predefined height along a direction parallel to the axis of rotation,
the first and second intake ports and the first and second discharge ports each have a height along the direction parallel to the axis of rotation,
the height of each port being smaller than the predefined height of each apex of the rotor.

6. The circulatory assistance system according to claim 5, wherein the stator further has a lower wall and an upper wall substantially perpendicular to the axis of rotation and each port extends at a distance from the lower wall and/or from the upper wall of the stator so that the side wall of the cavity is substantially continuous at the lower wall and/or at the upper wall of the stator.

7. The circulatory assistance system according to claim 4, wherein each port has a substantially rectangular section.

8. The circulatory assistance system according to claim 4, wherein, for one of the chambers, the distance between an area of the side wall immediately downstream of the intake port and an area of said side wall immediately upstream of the discharge port is greater than the sum of a distance between two apexes of the rotor and of a maximum dimension of an activated platelet, so as to form the clearance between the rotor and the stator.

9. The circulatory assistance system according to claim 4, wherein the stator further has an upper wall and a lower wall connecting the side wall of the cavity, a minimum distance between the upper wall and the rotor and between the lower wall and the rotor being greater than a maximum height of the rotor.

10. The circulatory assistance system according to claim 9, wherein the lower wall and the upper wall have surface irregularities configured to locally create a turbulent stream in the system.

11. The circulatory assistance system according to claim 8, wherein the rotor extends at a distance of at least 7 micrometers from the walls of the stator.
